(19) United States
(12) Patent Application Publication
(10) Pub. No.: US 2019/0200913 A1
(43) Pub. Date: Jul. 4, 2019

(54) MOVEMENT DISORDERS MONITORING AND TREATMENT SUPPORT SYSTEM FOR ELDERLY CARE USING EXTERNAL PRESSURE SENSITIVE DEVICE

(71) Applicant: VOCATIONAL TRAINING COUNCIL, Hong Kong (HK)

(72) Inventors: Raymond Kwan LAI, Hong Kong (HK); Ivan Man Chim TSE, Hong Kong (HK); Calvin Hoi Kok CHEUNG, Hong Kong (HK); Timothy Kin Sang LEE, Hong Kong (HK); Chi Pang LAM, Hong Kong (HK); Sing Yee NG, Hong Kong (HK); Eric Sai Lok LIU, Hong Kong (HK); Ching Ching CHEUNG, Hong Kong (HK); Leung CHIU, Hong Kong (HK); Hon Kong CHAN, Hong Kong (HK); Hung Keung TSE, Hong Kong (HK)

(21) Appl. No.: 15/856,927

(22) Filed: Dec. 28, 2017

(51) Int. Cl. A61B 5/00; A61B 5/11

(52) U.S. Cl. CPC A61B 5/4082; A61B 5/0022; A61B 5/1125; A61B 5/7282; A61B 5/742; A61B 5/486; A61B 2562/168; A61B 5/7267; A61B 5/7246; A61B 5/4836; A61B 2503/08; A61B 2562/0247; A61B 2562/028; A61B 5/7475

(57) ABSTRACT

Disclosed embodiments include a complete system and platform, and a method which allows for monitoring and supporting treatment for elderly care in clinic, home, and other normal daily environments. The system includes: (1) a pressure sensitive device for receiving force response from the elderly and convert the force response into digitalized pressure data, (2) a computing device for running a movement disorder assessment module to receive digitalized pressure data, display visual instructions and feedback, and process the digitalized pressure data for calculation of movement disorder scores, and (3) a cloud based digital diary to store, access and analyze the movement disorder scores, and generate reports on demand. The method will calculate the movement disorder scores based on the digitalized pressure data. A pressure sensitive device is also disclosed.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

### 1. Field of the Invention

This invention relates to monitoring and supporting treatment for elderly care by assessing, and recording of some symptoms of movement disorder, for example, rigidity and bradykinesia. Specifically, it relates to systems and devices for monitoring measurement of the symptoms of movement disorder using pressure sensitive device, a computing device such as tablet, a computational methodology to compute movement disorder scores and a cloud based digital diary. The scores will be uploaded to the cloud based digital diary for recording and assist clinicians to monitor and support treatment for elderly care.

### 2. Description of Related Art Including Information Disclosed Under 37 CFR 1.97 and 37 CFR 1.98

Majority of the existing movement disorder monitoring device use inertial sensor, for example the accelerometer and gyroscope to collect data such as speed and acceleration with respect to the movement and position of a human body and base on such data to measure the seriousness of movement disorder. Such monitoring device can be broadly divided into two categories, namely activity monitoring and inertia monitoring. They both have their advantages and disadvantages in monitoring and measuring the movement disorder but the data obtained is usually mixed with other data associated with voluntary movement such that errors are unavoidable.

US patent (Patent No. 0098608 A1) invents an automated method of determining a kinetic state of a person by collecting data from accelerometer worn on an extremity of the person. Data of significant movement is needed to be will be extracted first from the accelerometer data. Such data will be further band-passed to extract the low frequency component related to movement disorder. Although this method only requires a person wear a small device which is similar to the watch or fitness bracelet during the daily time, the data obtained is mixed with data of voluntary movement and are prone to errors.

US patent (U.S. Pat. No. 5,293,879) invent a device and method to collect high resolution data of the person's movement data. These measuring devices are bigger in size and equipped with high-quality inertial sensor to survey the movement of the elderly. The sensors of the device can sense and record six movement degrees of freedom (three linear axes and three rotation axes). Besides, such monitoring device can save the resulting data in their storage for later analysis, or can send those results via wireless or wireline communication module to nearby computer or the recording equipment for further analysis. These devices are useful to obtain all symptoms of the movement disorder of the person. However, as the size of the device is big, it is generally not suitable for non-clinical environment or long-time monitoring purposes.

The contemporary system and method for movement disorder monitoring and assessment lacks mobility, interactivity, scheduling and collaboration capability. In view of this, the elderly needs a system and method which enable them to perform self-assessment in a scheduled manner such that it is no longer necessary for the elderly to wear a sensor device continuously and it is enabled the remote inspection by clinicians for monitoring or other clinical purposes. In addition, the assessment should be designed in a specific way such that the resulting data is more accurate, reliable and free from interruption or disturbance of other movement data generated from daily normal movement. Further, the assessment should be conducted in an easy, interactive and game like manner such that the overall training cost of the assessment will be reduced as the elderly could do the test in a correct way even for the first time.

Interactive Environment for Movement Disorder Assessment

In the present invention, movement disorder monitoring and assessment will be achieved by asking the elderly to participate in an interactive assessment environment. For example, visual instructions can be put on a display of computing device and invite the elderly to perform certain actions via an external device in accordance with those visual instructions. By collecting data from external device during the elderly is performing the assessments, the degree of movement disorder can be assessed.

In current medical practice and clinical trial, the degree of movement disorder is evaluated using rating scales by the trained physician or neurologist. However, such clinical evaluation is rough, subjective and temporary. These evaluation methodologies also put the elderly under pressure and is insensitive to the subtle emotional change of the elderly and most importantly, these kinds of evaluation are subjective and can only be conducted by trained personnel. In the market, some tools such as patient diary can be used by the elderly as a self-reporting tool to record their entire day movement and allow the elderly to report the records to their clinician later. However, these methods are often inaccurate, incomplete and are also subjected to prejudice of sensation and recollection of the elderly.

With the advancement of technology, small but powerful computing device such as tablet becomes commonly available in every household. Tablet is small in physical size, low power consumption, equipped with wireless interconnection capabilities and high computational power. They are ideal platforms for creating an interactive assessment environment to monitor and support treatment for elderly care by hosting and execution of monitoring and assessment software application and interfacing with low cost, low power consumption and small external sensors to receive movement data from the elderly. Such monitoring and assessment solution is low cost, not subjected to any restrictions of laboratory space and can be used by the elderly for self-assessment purpose.

Some movement disorder symptom such as rigidity refers to an increase in resistance to movement. Another symptom of movement disorder, bradykinesia, which will primarily slow down the movement of the elderly because of inability to produce a normal level of working force. Under these movement disorder symptoms, the elderly generally takes longer time to perform an action even if such action is a routine and normal work. When the elderly performs a series of forceful actions in a designed assessment environment, the parameters obtained from such assessment will show lower response than using the parameters obtained from the one without these symptoms. Therefore, the current invention invites the elderly repeatedly performs a forceful action such as squeezing a rubber bulb in accordance with visual instructions shown on the screen, and the force exerted on the rubber bulb will be recorded, processed and calculated to give out movement disorder scores for monitoring and supporting treatment for elderly care.

The current invention of device and system intends to provide a small and affordable solution for the elderly to perform a self-monitoring and assessment of movement disorder without undue pressure in an interactive environment.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF SUMMARY OF THE INVENTION

An embodiment of the present invention provides a method and system for monitoring and supporting treatment for elderly care; a movement disorder assessment module running on a computing device shows visual instructions and feedback on the computing device's screen to invite the elderly to control a visual object shown on the computing device's screen via an external pressure sensitive device. The pressure sensitive device will convert the exerted force for controlling the object into digitalized pressure data and sent to a computing device for further handling. The computing device will process and analyze the received digitalized pressure data and calculate movement disorder scores. The resulting movement disorder scores will be uploaded and stored into a cloud based digital diary. The cloud based digital diary provides computer user interfaces to access and generate report of those scores on demand.

### BRIEF DESCRIPTION OF THE SEVERAL VIEWS OF THE DRAWINGS

FIG. 1 illustrates a block diagram representing the basic components of an embodiment of the general system for monitoring and supporting treatment for elderly care.

FIG. 2 illustrates a diagram for controlling the length of a bar so that the two ends of the bar follow closely the two moving vertically sinusoidal curve.

FIG. 3 illustrates a diagram for controlling the altitude of the helicopter so that the helicopter follows closely the laterally moving sinusoidal curve.

FIG. 4 illustrates a flow diagram of the squeeze bulb test.

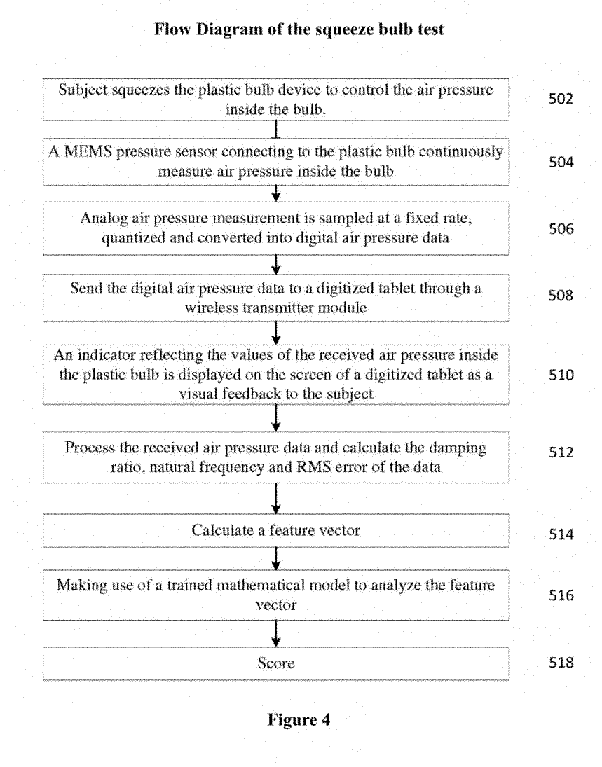

FIG. 5 illustrates a graphical summary report based on movement disorder scores.

FIG. 6 illustrates a functional block diagram of pressure bulb using resistor network.

### DETAILED DESCRIPTION OF THE INVENTION

While the present invention will be described more fully it is to be understood at the outset of the description which follows that persons of skill in the appropriate arts may modify the invention herein described while still achieving the favourable results of this invention. Accordingly, the description which follows is to be understood as being a broad, teaching disclosure directed to persons of skill in the appropriate arts, and not as limiting upon the present invention.

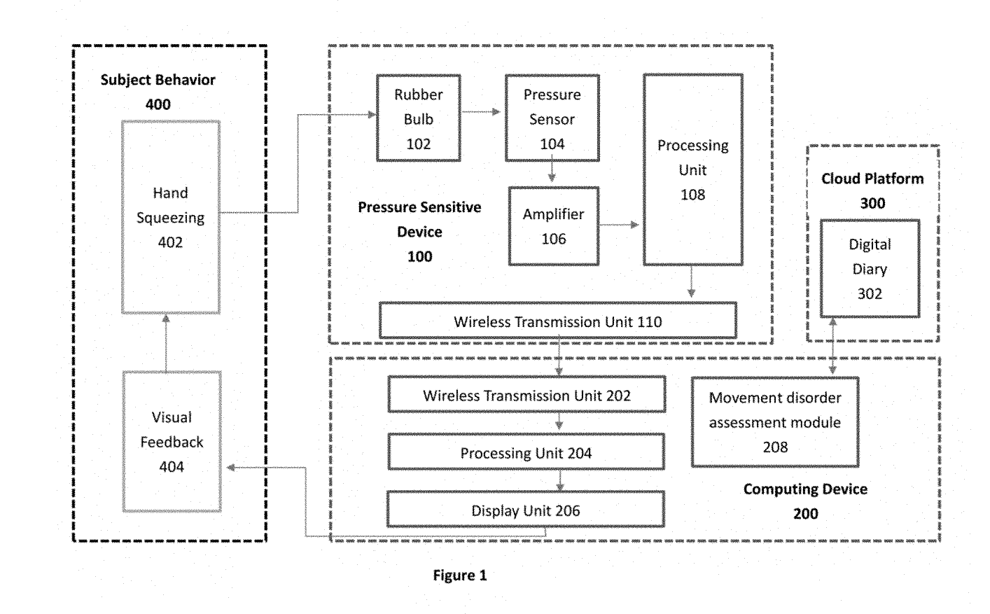

According to one embodiment, as shown in FIG. 1, the system for monitoring and supporting treatment for elderly care comprises: one or more pressure sensitive devices 100, one or more computing devices 200 connected to one or more cloud platforms 300, and a movement disorder assessment module 208 running on computing devices 200 to process digitalized pressure data collected from pressure sensitive devices 100 and to calculate movement disorder scores, and cloud based digital diary 302 running on a cloud platform 300 to store such scores and provide computer user interfaces for access, research and analysis.

According to one embodiment, as shown in FIG. 1, the pressure sensitive device 100 is a lightweight and small device comprising: a rubber bulb 102 as external physical interface to receive force response; a pressure sensor 104 to survey the force response; an amplifier 106 configured to amplify and generate analog electrical signal from the pressure sensor 104; a processing unit 108 configured for device control, device status, device communication and digitization of analog electrical signal; and a wireless transmission unit 110 for communicating with the computing device 200.

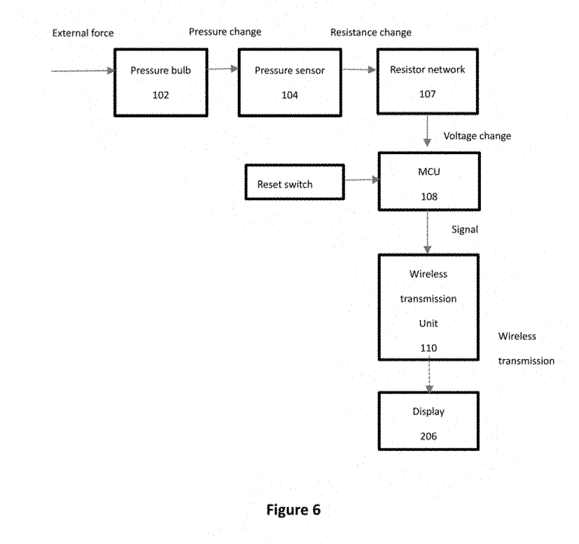

According to another embodiment, as shown in FIG. 6, the pressure sensitive device 100 is a lightweight and small device comprising: a rubber bulb 102 as external physical interface to receive force response; a pressure sensor 104 to survey the force response; an resistor network 107 configured to generate analog electrical signal received from the pressure sensor 104; a processing unit 108 configured for device control, device status, device communication and digitization of analog electrical signal; and a wireless transmission unit 110 for communicating with the computing device 200.

In one embodiment, a physical external interface for receiving force response from the elderly including a rubber bulb 102 connected to a device's pressure sensor 104. In one embodiment, the pressure sensor 104 can be any MEMS pressure sensor and encapsulated in all kind of housings with board pressure range, either absolute or differential. The pressure sensor 104 can sense the pressure generated by the force exerted on a rubber bulb 102 and convert the pressure into analog electrical signal where the amplitude of the electrical signal depends upon the degree of pressure applied. The pressure sensor 104 and the processing unit 108 is bridged by an electronic component. In one embodiment, the electronic component is an amplifier 106 which is a type of quad operational amplifier to amplify the analog electrical signal generated from the pressure sensor 104. In another embodiment, the electronic component is a resistor network 107 which can sense the change of resistance generated from the pressure sensor 104 and output the analog electrical signal. In one embodiment, a processing unit 108 includes a microcontroller which digitalizes the analog electrical signal into digitalized pressure data and send the digitalized pressure data via a wireless transmission unit 110 to the computing device 200. In one embodiment, the wireless transmission unit 110 is designed to communicate with a computing device 200 to transmit digitalized pressure data, using WIFI technology. In another embodiment, the wireless transmission unit 110 is designed to communicate with a computing device 200 to transmit digitalized pressure data, using Bluetooth technology. In another embodiment, the wireless transmission unit 110 is designed to communicate with a computing device 200 to transmit digitalized pressure data, using RF technology and the like.

In one embodiment, a computing device 200 includes a wireless transmission unit 202, a processing unit 204 and a display unit 206 to provide computational power for running a movement disorder assessment module 208 and wireless interconnectivity for receiving digitalized pressure data from a pressure sensitive device 100 and uploading movement disorder scores to a cloud platform 300 running a cloud based digital diary 302. In one embodiment, the wireless transmission unit 202 is designed to communicate with a pressure sensitive device 100 to receive digitalized pressure data, using WIFI technology. In another embodiment, the wireless transmission unit 202 is designed to communicate with a pressure sensitive device 100 to receive digitalized pressure data, using Bluetooth technology. In another embodiment, the wireless transmission unit 202 is designed to communicate with a pressure sensitive device 100 to receive digitalized pressure data, using RF technology and the like.

In one embodiment, a movement disorder assessment module 208 provides visual instruction on the computing device's 200 display unit 206 to invite the elderly to respond by hand squeezing 402 on the rubber bulb 102 of a pressure sensitive device 100 to control the air pressure inside the bulb 502, pressure sensor 104 continuously measure air pressure and convert it into analog electrical signal 504.

The pressure sensitive device 100 sends the digitalized pressure data to a computing device 200 running a movement disorder assessment module 208 via the wireless transmission unit 110. The movement disorder assessment module 208 will process the digitalized pressure data and feedback to the elderly on the computing device's 200 display unit 206.

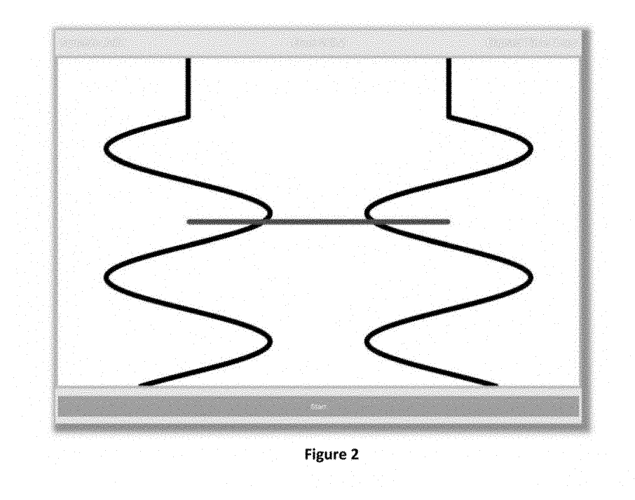

According to a preferred embodiment, as show in FIG. 2, the visual instruction consists of an object which is a bar located at the centre and enclosed by a pathway created from two simultaneously moving sinusoidal pattern. The sinusoidal pattern will be continuously moving and the elderly has to control the length of the bar by hand squeezing 402 the rubber bulb 102 of the pressure sensitive device 100. The length of the bar will be proportional to the force exerted on the rubber bulb 102 and the elderly has to control and keep the ends of the bar close to and within the pathway created by the sinusoidal patterns. The digitalized pressure data will be sent to the movement disorder assessment module 208 running on a computing device 200 for processing and analysing the digitalized pressure data, and providing visual feedback 404 to the elderly.

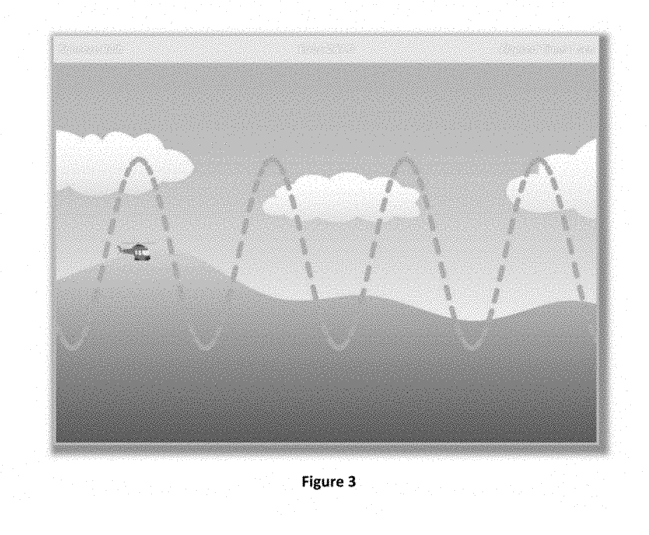

According to another preferred embodiment, as shown in FIG. 3, visual instruction consists of a helicopter and a moving sinusoidal pattern. The sinusoidal pattern will be continuously moving and the elderly has to control and keep the latitude of the helicopter close to a pathway created by the sinusoidal pattern. The elderly has to control the latitude of the helicopter by hand squeezing 402 on the rubber bulb 102 of the pressure sensitive device 100 and the vertical position of helicopter will be directly proportional to the force exerted on the rubber bulb 102. The digitalized pressure data will be sent to the movement disorder assessment module 208 running on a computing device 200 for processing and analysing the digitalized pressure data, and providing visual feedback 404 to the elderly.

Movement disorder assessment module runs a computational method to analyze the digitalized pressure data and compute movement disorder scores for monitoring and supporting treatment for elderly care. In one embodiment, the system calculates scores for some symptoms of movement disorder, for example, rigidity and bradykinesia. In one embodiment, the computational method processes the digitalized pressure data and calculate the damping ratio, natural frequency and RMS error of such data 512 using any contemporary mathematical model such as standard discrete second-order linear dynamical system. Kernel principal component analysis (KPCA) or kernel discriminant analysis (KDA) or other contemporary analysis will be used by the computational method for further feature extraction or dimensionality reduction. The computational method further calculates a feature vector 514 and making use of a trained mathematical model to analyse the feature vector 516 to calculate the movement disorder scores 518.

The movement disorder scores for monitoring and supporting treatment for elderly care will be uploaded by movement disorder assessment module 208 to a cloud based digital diary's 302 database via the wireless transmission unit 202 for storing. In one embodiment, user name and password of the elderly can be configured in the computing device 200 and is used to authenticate and login to a cloud based digital diary 302 using wireless connectivity such as Internet enabled WIFI connection. Once the authentication is successful, the movement disorder scores can be securely uploaded to the cloud based digital diary 302.

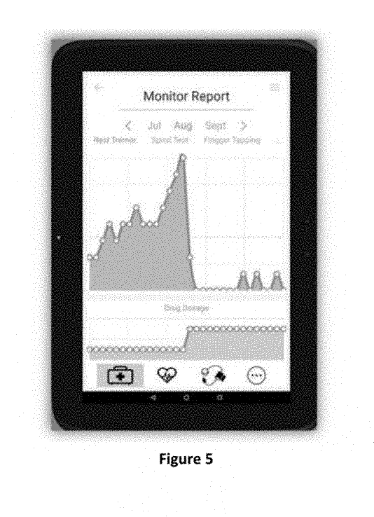

The cloud based digital diary 302 provides data access and reporting tools to clinicians or other relevant personnel. In one embodiment, the cloud based digital diary 302 provides computer user interfaces to the clinicians or other relevant personnel to access movement disorder scores and generate report through the computer user interfaces. According to one embodiment, as shown in FIG. 5, the cloud based digital diary 302 can generate graphical summary report by extracting and processing movement disorder scores such that clinicians can understand the effect of the prescribed drug type and its dosage on the elderly. Thus, the clinicians could adjust drug type or dosage or the drug combination prescribed based on the summary report accordingly in order to minimize the chance of drug over-dose or under-dose.

The cloud based digital diary 302 also provides function to schedule assessment. In one embodiment, clinicians can input schedule via computer user interfaces of the cloud based digital diary 302 and the movement disorder assessment module 208 can download the schedule from the cloud based digital diary 302 each day and alert the elderly to participate the assessment using visual alarm displayed in the display unit 206.

We claim:

1. A system for monitoring and supporting treatment for elderly care comprising:
a) a pressure sensitive device;
b) a computing device;
c) a movement disorder assessment module operable on the computing device; and
d) a cloud based digital diary.

2. The system for monitoring and supporting treatment for elderly care of claim 1, the said pressure sensitive device comprising:
a) a physical interface configured to receive force response from the elderly,
b) a pressure sensor;
c) a processing unit;
d) an electronic component configured to bridge between the said pressure sensor and the said processing unit; and
e) a wireless transmission unit.

3. The system for monitoring and supporting treatment for elderly care of claim 2, wherein the said physical interface includes a rubber bulb and tube for receiving the elderly's force response and passing the force response as pressure to the said pressure sensor via the tube.

4. The system for monitoring and supporting treatment for elderly care of claim 2, wherein the said pressure sensor is a MEMS pressure sensor and configured to sample pressure at a fixed rate, quantize and convert the pressure into analog electrical signal.

5. The system for monitoring and supporting treatment for elderly care of claim 2, wherein the said electronic component is an instrumentation amplifier to amplify and output analog electrical signal to the said processing unit.

6. The system for monitoring and supporting treatment for elderly care of claim 2, wherein the said electronic component is a resistance network to output analog electrical signal to the said processing unit.

7. The system for monitoring and supporting treatment for elderly care of claim 2, wherein the said processing unit configured to receive the analog electrical signal and digitalize such signal into digitalized pressure data.

8. The system for monitoring and supporting treatment for elderly care of claim 2, wherein the said wireless transmission unit includes a wireless interface and is configured for communicating with the said computing device to transmit the digitalized pressure data.

9. The system for monitoring and supporting treatment for elderly care of claim 8, wherein the said wireless interface is a WIFI interface or Bluetooth interface or RF interface.

10. The system for monitoring and supporting treatment for elderly care of claim 1, wherein the said computing device comprising: a second wireless transmission unit; a second processing unit; a display unit; and a movement disorder assessment module.

11. The system for monitoring and supporting treatment for elderly care of claim 10, wherein the said wireless transmission unit includes a wireless interface to provide wireless connectivity to the said pressure sensitive device.

12. The system for monitoring and supporting treatment for elderly care of claim 11, wherein the said wireless interface is a WIFI interface or Bluetooth interface or RF interface.

13. The system for monitoring and supporting treatment for elderly care of claim 10, wherein the said processing unit configured to provide computational power for running the said movement disorder assessment module.

14. The system for monitoring and supporting treatment for elderly care of claim 10, wherein the said display unit configured to display visual instructions for assessment and visual feedback based on the digitalized pressure data.

15. The system for monitoring and supporting treatment for elderly care of claim 10, wherein the said movement disorder assessment module configured to provide visual instructions and feedback via the said display unit, process the digitalized pressure data receiving from the said pressure sensitive device and calculate movement disorder scores using a plurality of computational methods.

16. The system for monitoring and supporting treatment for elderly care of claim 1, wherein the said cloud based digital diary configured to receive and store movement disorder scores uploaded from the said movement disorder assessment module and provide computer user interfaces for access and analyze such data.

17. A method for monitoring and supporting treatment for elderly care, comprising the steps of:
scheduling an assessment by inputting date and time into a cloud based digital diary;
instructing the elderly to participate an assessment;
displaying visual instructions and an object to be controlled by the elderly;
controlling the said object by exerting force on rubber bulb of a pressure sensitive device;
acquiring digitalized pressure data continuously from the said pressure sensitive device;
calculating damping ratio, natural frequency and root mean square error based on the digitalized pressure data;
calculating feature vectors;
calculating movement disorder scores based on the said feature vectors;
storing the said movement disorder scores into a cloud based digital diary; and
generating a summary report.

18. The method according to claim 17, wherein the step of instructing the elderly to participate an assessment is accomplished by showing a visual alert on a computing device in accordance with date and time of the scheduled assessment.

19. The method according to claim 17, wherein the said visual instructions is a pathway created from a moving repeated pattern or a boundary created from a plurality of moving repeated patterns.

20. The method according to claim 19, wherein the said moving repeated pattern is sinusoidal.

21. The method according to claim 17, wherein the said object is a bar and the elderly has to control the length of the bar so that two ends of the bar follow closely the boundary formed between two simultaneously moving repeated pattern.

22. The method according to claim 17, wherein the said object is a helicopter and the elderly has to control the altitude of the helicopter so that the helicopter follows closely the pathway created by a moving repeated pattern.

23. The method according to claim 17, wherein the step of controlling the said object by exerting force on rubber bulb of the said pressure sensitive device, the exerting force is transformed into digitalized pressure data to control the length or the altitude of the said object.

24. The method according to claim 21, wherein the length or the altitude of the object is calculated from magnitude of the digitalized pressure data.

25. The method according to claim 17, wherein the said damping ratio, the said natural frequency and the said root mean square error is calculated from the digitalized pressure data.

26. The method according to claim 17, wherein the feature vector is calculated from the said root mean square errors, the said damping ratio, and the said natural frequency.

27. The method according to claim 17, wherein the said movement disorder scores will be calculated from the said feature vector by making use of a trained mathematical model.

28. The method according to claim 17, wherein the said summary report can be generated using computer user interface of cloud based digital diary such that clinicians could understand and be presented the conditions of the elderly according to scheduled date and time of the assessment.

29. The method according to claim 28, wherein the said summary report can be further configured to show correlation between the elderly's movement disorder scores and their prescribed drug types and dosage.

30. The method according to claim 29, wherein the correlation enables clinicians to adjust drug type and dosage or the combination of drugs and dosage prescribed to the elderly for minimizing the chance of drug over-dose or under-dose.
